(19) United States
(12) Patent Application Publication
(10) Pub. No.: US 2015/0157302 A1
(43) Pub. Date: Jun. 11, 2015

(54) Clinical Specimen Sampler and Method thereof

(71) Applicant: National Tsing Hua University, Hsin Chu City (TW)

(72) Inventors: Shu-Ting FAN, New Taipei City (TW); Hsi-Kai WANG, Taipei City (TW); Hong-Ren Lin, New Taipei City (TW); Ting-Chang Chang, Taoyuan County (TW); Chia-Chi Wu, Yilan City (TW); Chao-Min Cheng, New Taipei City (TW)

(73) Assignee: National Tsing Hua University, Hsin Chu City (TW)

(21) Appl. No.: 14/258,020

(22) Filed: Apr. 22, 2014

(30) Foreign Application Priority Data
Dec. 10, 2013 (TW) 102145462

(51) Int. Cl. A61B 10/00; A61B 17/00; A61F 13/38

(52) U.S. Cl. CPC A61B 10/0045; A61F 13/38; A61B 17/00234; A61B 2017/00287; A61B 2010/0074

(57) ABSTRACT

The present invention discloses a clinical specimen sampler, including a filtering device; and a sampling device. The sampling device includes a sampling adsorber and a rod. The sampling adsorber is connected with one end of the rod, and the sampling adsorber is encompassed in the filtering device. The sampling adsorber is utilized to absorb the body fluid secreted by the intima of an organ, and the filtering device is employed to prevent the mucosal tissue of the intima of the organ from entering the sampling adsorber and enable the body fluid secreted by the intima of the organ to enter the sampling adsorber, so as to be absorbed by the sampling adsorber.

### CROSS-REFERENCE TO RELATED APPLICATIONS

This present application claims priority to TAIWAN Patent Application Serial Number 102145462, filed on Dec. 10, 2013, which is herein incorporated by reference.

### TECHNICAL FIELD

The present invention relates to clinical specimen sampling, and more particularly to a clinical specimen sampler and a method thereof.

### BACKGROUND OF RELATED ART

Recently, the common clinical specimen sampler, for example the endometrial cancer specimen sampler, generally includes suction tubes for uterus cavity, uterus cavity brushes, plastic spatulas for uterus cavity, uterus cavity currettes, etc. The way to utilize them is to place them into the uterus cavity, and suck or currette the surface of the endomembrane to obtain the endomembrane cell and its secretion, so as to further process and test the endomembrane cell and its secretion to find cancel cells. The positive rate thereof may reach more than 90%. This method can be executed at the clinic, and no operation or anesthesia is needed, but the method can be utilized only for screening. A final definite diagnosis still must be made according to pathological examination results.

However, the endomembrane cell and its secretion obtained by the traditional endometrial cancer specimen sampler mentioned above often mix with each other. Therefore, if it is desired to test the secretion, the endomembrane cell and the secretion need to be separated first. Thus, the pretreatment procedure before directly testing the secretion will be complicated. Besides, the traditional endometrial cancer specimen sampler mentioned above often hurts the test subject or makes the test subject feel quite uncomfortable when sucking or curretting the surface of the endomembrane to obtain the endomembrane cell and its secretion, which renders the test subject to refuse the usage of the traditional endometrial cancer specimen sampler.

Therefore, there is still a need for a technical solution which can solve the shortcomings of the traditional clinical specimen sampler.

### SUMMARY

To overcome the shortcomings of the traditional clinical specimen sampler, for instance the endometrial cancer specimen sampler, the present invention provides a novel clinical specimen sampler, which can enhance the feeling of comfort of the test subject when used and simplify the pretreatment procedure before testing.

In one aspect, the present invention discloses a clinical specimen sampler including a filtering device; and a sampling device, the sampling device comprising a sampling adsorber and a rod, the sampling adsorber being connected with one end of the rod, the sampling adsorber being encompassed in the filtering device, wherein the sampling adsorber is utilized to absorb a body fluid secreted by an intima of an organ and the filtering device is employed to prevent a mucosal tissue of the intima of the organ from entering the sampling adsorber and enable the body fluid secreted by the intima of the organ to enter the sampling adsorber, so as to be absorbed by the sampling adsorber.

In one embodiment, the filtering device may be bag-shaped. In one embodiment, for instance, not for limiting the present invention, a binding wire or a quickly detachable fixing device may be disposed around a bag opening of the filtering device to fix the filtering device onto the sampling adsorber of the sampling device. In one embodiment, the filtering device may include a filter screen. In one embodiment, the material of the filtering device may include gauze, non-woven fabric paper towel, non-woven fabric bandage or the equivalent thereof or other material which can filter the mucosa and the secretion tissue. In one embodiment, the material of the filtering device may preferably be non-woven fabric paper towel.

In one embodiment, the material of the sampling adsorber may include sponge block, cotton ball or the equivalent thereof or other material which can adsorb the body fluid. In one embodiment, the material of the sampling adsorber may preferably be cotton ball. In one embodiment, the material of the rod may include wooden rod, bamboo rod or plastic rod. In one embodiment, the clinical specimen sampler of the present invention may further include a lubricant coated on the outer surface of the filtering device to enhance the comfortability. In one embodiment, the size of the mesh of the filtering device may be at least smaller than the size of the mucosal tissue.

In another aspect, the present invention discloses a clinical specimen sampling method employing the aforementioned clinical specimen sampler, the method including rendering the sampling adsorber encompassed in the filtering device to contact the surface of the intima of the organ of a test subject to adsorb the body fluid secreted by the intima of the organ; loosening the binding wire or the quickly detachable fixing device and separating the sampling device and the filtering device, such that the sampling device adsorbed with the body fluid is separated from the filtering device adhered with the mucosal tissue; placing the sampling adsorber in an upper centrifuge tube of a centrifuge tube and separating the sampling adsorber from the rod; adding a buffer solution into the upper centrifuge tube and combining the upper centrifuge tube with a lower centrifuge tube to form the centrifuge tube; placing the combined centrifuge tube in a centrifuge to perform centrifugation procedure; and separating the upper centrifuge tube from the lower centrifuge tube to obtain the body fluid mixed with the buffer solution in the lower centrifuge tube.

One advantage of the present invention is that the clinical specimen sampler of the present invention can sample the body fluid secreted by the intima of the organ, and the sampled body fluid will not be mixed with the substances other than the body fluid, such as the mucosal tissue, such that no additional pretreatment steps are needed to remove the mucosal tissue when performing testing.

Another advantage of the present invention is that the clinical specimen sampler of the present invention is quite convenient to the user when used and is cheap.

Still another advantage of the present invention is that the lubricant is coated on the outer surface of the filtering device of the clinical specimen sampler of the present invention, such that the comfortability to the test subject is enhanced when applying the clinical specimen sampler to the test subject.

Yet another advantage of the present invention is that the clinical specimen sampler of the present invention only contacts the surface of the intima of the organ of the test subject, and needs not to currette or suck the surface of the intima of the organ under negative pressure, so as to decrease the feeling of hurt or uncomfort to the test subject.

These and other advantages will become apparent from the following description of preferred embodiments taken together with the accompanying drawings and the appended claims.

### BRIEF DESCRIPTION OF THE DRAWINGS

The present invention may be understood by some preferred embodiments and detailed descriptions in the specification and the attached drawings below.

FIG. 1 illustrates a diagram of a clinical specimen sampler of the present invention in accordance with one embodiment of the present invention;

FIG. 2 illustrates a flow chart of a clinical specimen sampling method of the present invention in accordance with one embodiment of the present invention;

FIG. 3 illustrates a flow chart of the clinical specimen sampling method of the present invention in accordance with one embodiment of the present invention;

FIG. 4 illustrates a flow chart of the clinical specimen sampling method of the present invention in accordance with one embodiment of the present invention; and

FIG. 5 illustrates a flow chart of the clinical specimen sampling method of the present invention in accordance with one embodiment of the present invention.

### DETAILED DESCRIPTION

The present invention will now be described with the preferred embodiments and aspects and these descriptions interpret structure and procedures of the present invention only for illustrating but not for limiting the Claims of the present invention. Therefore, except the preferred embodiments in the specification, the present invention may also be widely used in other embodiments.

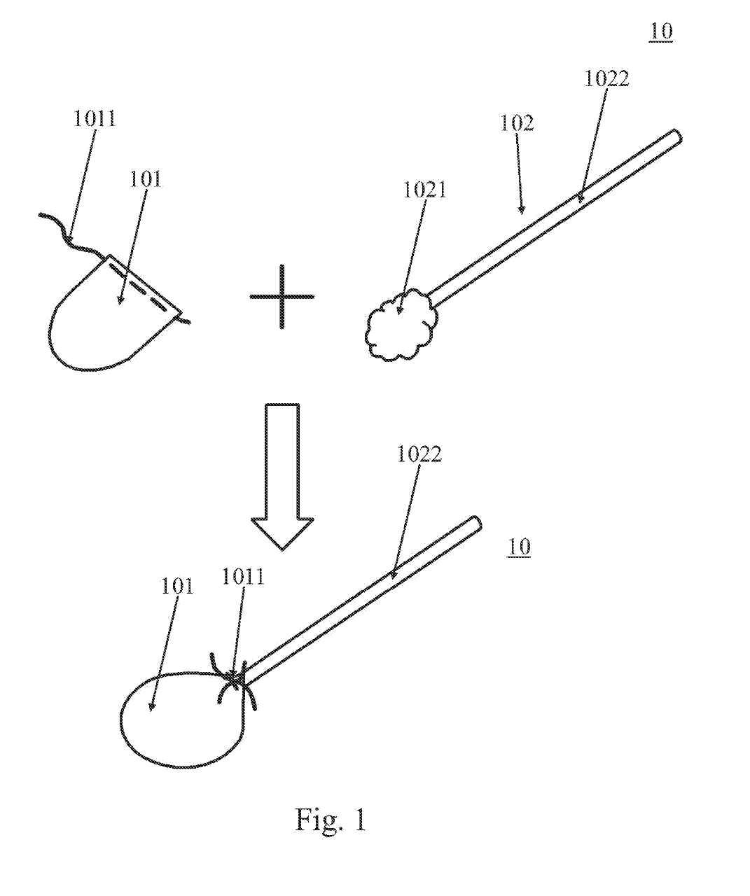

The present invention provides a clinical specimen sampler and a method thereof. The clinical specimen sampler of the present invention may be applied to any diseases which can be tested via body fluids, for example but be not limited to, the endometrial cancer, or may be utilized to detect the physiological condition of the test subject, for instance but be not limited to, the ovulation cycle of woman. In one embodiment of the present invention, as shown in FIG. 1, the clinical specimen sampler 10 of the present invention may include a filtering device 101 and a sampling device 102. In one embodiment, the filtering device 101 may be a filter screen. In one specific embodiment, the material of the filtering device 101 may include but be not limited to gauze, non-woven fabric paper towel, non-woven fabric bandage or the equivalent thereof or other material which can filter the mucosa and the secretion tissue, so as to filter the substance except the body fluid, such as the mucosal tissue, prevent the mucosal tissue from entering the sampling adsorber of the sampling device, and enable the body fluid secreted by an intima of an organ to enter the sampling adsorber of the sampling device, so as to be absorbed by the sampling adsorber. In a preferred embodiment, the material of the filtering device 101 may preferably be the non-woven fabric paper towel because it is much comfortable to the test subject. The size of the mesh of the filtering device may be at least smaller than the size of the mucosal tissue, so as to filter out the mucosal tissue. In one embodiment, the filtering device 101 may be bag-shaped. In one embodiment, for example, not for limiting the present invention, a binding wire 1011 or a quickly detachable fixing device may be disposed around the bag opening of the filtering device 101 to fix the filtering device 101 onto the sampling adsorber (which will be described in detail hereinafter) of the sampling device 102. In one embodiment, as shown in FIG. 1, the sampling device 102 may include a sampling adsorber 1021 and a rod 1022. The sampling adsorber 1021 is connected with one end of the rod 1022. In one embodiment, the material of the sampling adsorber 1021 may include but be not limited to sponge block, cotton ball or the equivalent thereof or other material which can adsorb body fluid. In one preferred embodiment, the material of the sampling adsorber 1021 may preferably be cotton ball. The sampling adsorber 1021 can adsorb the body fluid secreted by the intima of an organ, for instance but be not limited to, the endometrium, the inner membrane of the oral cavity, so as to perform disease testing or physiological condition testing while the rod 1022 can be held by the user. In one embodiment, the material of the rod 1022 may include but be not limited to wooden rod, bamboo rod, plastic rod or the equivalent thereof. In one embodiment, a special physically separable design, for example but be not limited to a breaking notch, may be formed on the rod 1022, such that the user can apply force to the breaking notch to break the rod easily and separate the sampling adsorber 1021 and the rod 1022 easily, so as to further process the sampling adsorber 1021. The size of the filtering device 101 is slightly larger than the size of the sampling adsorber 1021 of the sampling device 102, such that the sampling adsorber 1021 can be encompassed in the filtering device 101 and the binding wire 1011 or the quickly detachable fixing device can be employed to tightly fix or bind the filtering device 101 to form the clinical specimen sampler 10 as shown in FIG. 1. In one embodiment, a lubricant, for instance but be not limited to, vaseline may be coated on the outer surface of the filtering device 101 encompassing the sampling adsorber 1021 to enhance the comfortability to the test subject when applying the clinical specimen sampler 10 to the test subject.

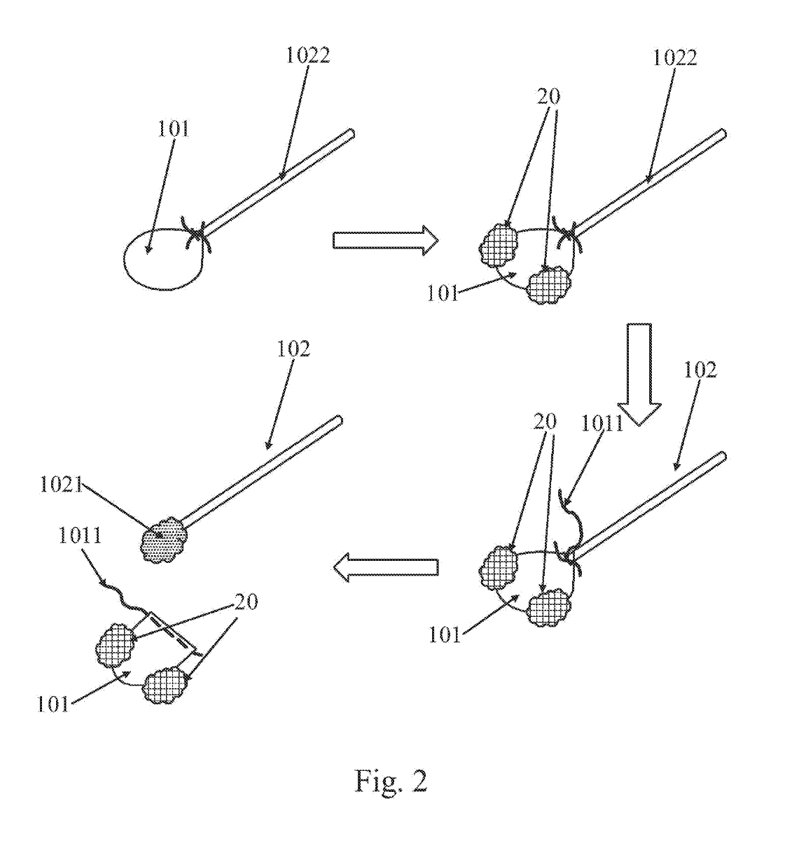

Please refer to FIG. 2, which illustrates a clinical specimen sampling method. First, the user may hold the rod 1022 and render the sampling adsorber encompassed in the filtering device 101 to contact the surface of the intima of the organ, for instance the endometrium, the inner membrane of the oral cavity, etc of the test subject, to adsorb the body fluid secreted by the intima of the organ. While the sampling adsorber adsorbs the body fluid secreted by the intima of the organ, some substances except the body fluid, for example the mucosal tissue 20, may usually be adhered to the filtering device 101. These mucosal tissues 20 might be unnecessary for the disease testing or physiological condition testing and cannot pass the filtering device 101 to enter the sampling adsorber because the size of these mucosal tissues 20 is larger than the size of the mesh of the filtering device 101. Subsequently, as shown in FIG. 2, the user may loosen the binding wire 1011 or the quickly detachable fixing device and separate the sampling device 102 and the filtering device 101, such that the sampling device 102 adsorbed with the body fluid can be separated from the filtering device 101 adhered with the mucosal tissue 20. The sampling adsorber 1021 of the sampling device 102 adsorbed with the body fluid may be further processed to obtain the body fluid therein and perform the disease testing and/or the physiological condition testing while the filtering device 101 adhered with the mucosal tissue 20 may be disposed of or be utilized to perform other analyses. Therefore, the clinical specimen sampler of the present invention can sample the body fluid secreted by the intima of the organ, and the sampled body fluid will not be mixed with the substances other than the body fluid, such as the mucosal tissue, such that no additional steps are needed to remove the mucosal tissue when performing testing.

In one embodiment, the sampling adsorber 1021 of the sampling device 102 adsorbed with the body fluid may be further processed. For instance, the sampling adsorber 1021 adsorbed with the body fluid may be immerged in a buffer solution to render the body fluid to enter the buffer solution. The buffer solution with the body fluid therein may be further utilized to test whether the test subject has a specific disease, for instance endometrial cancer, etc or to test the physiological condition, for example but be not limited to the ovulation cycle of woman, of the test subject by employing diagnostic devices, for example but be not limited to, paper-based diagnostic devices, lateral flow immunoassay devices, cotton-based diagnostic devices and so on.

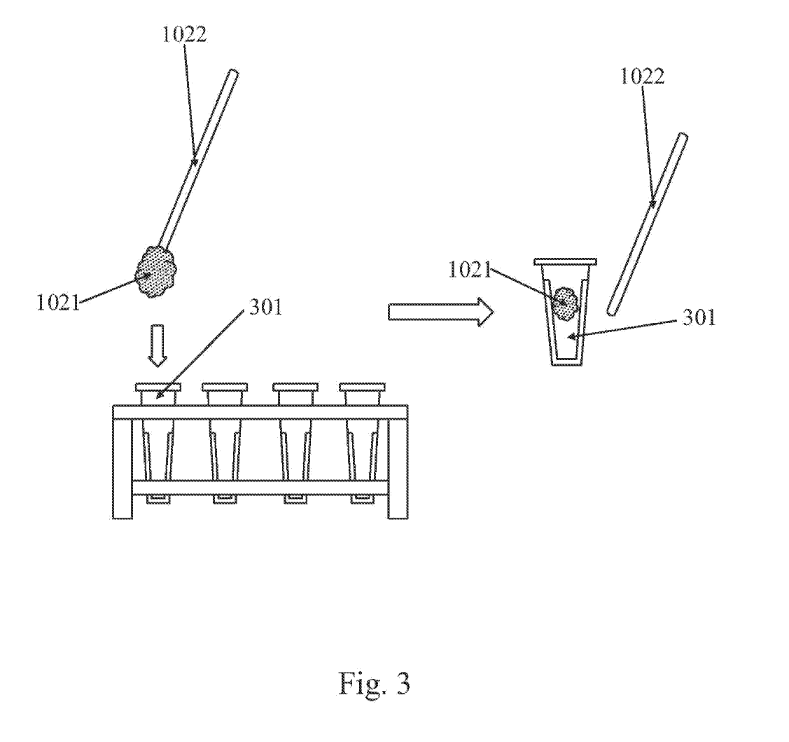

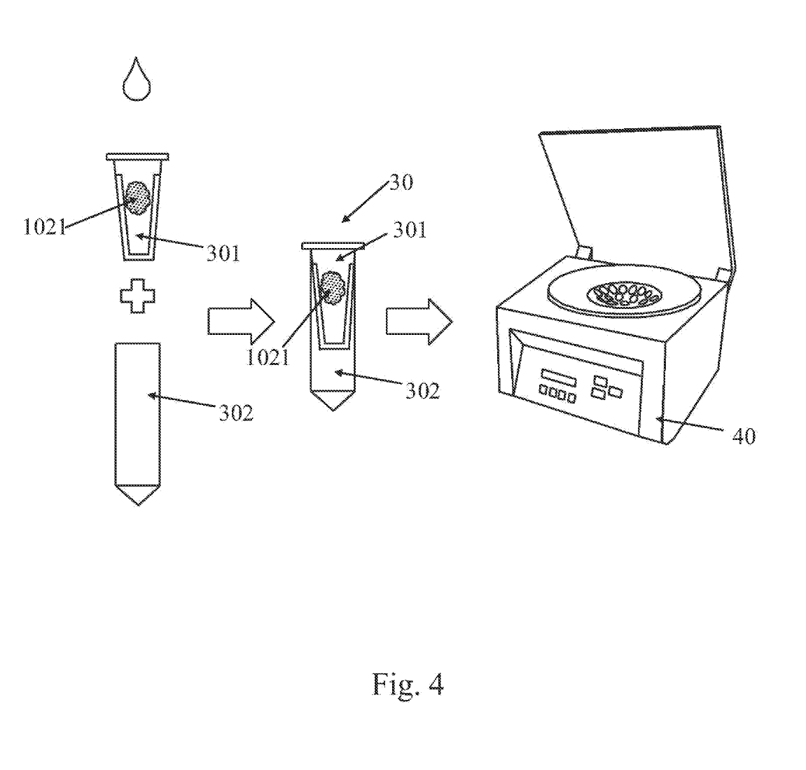

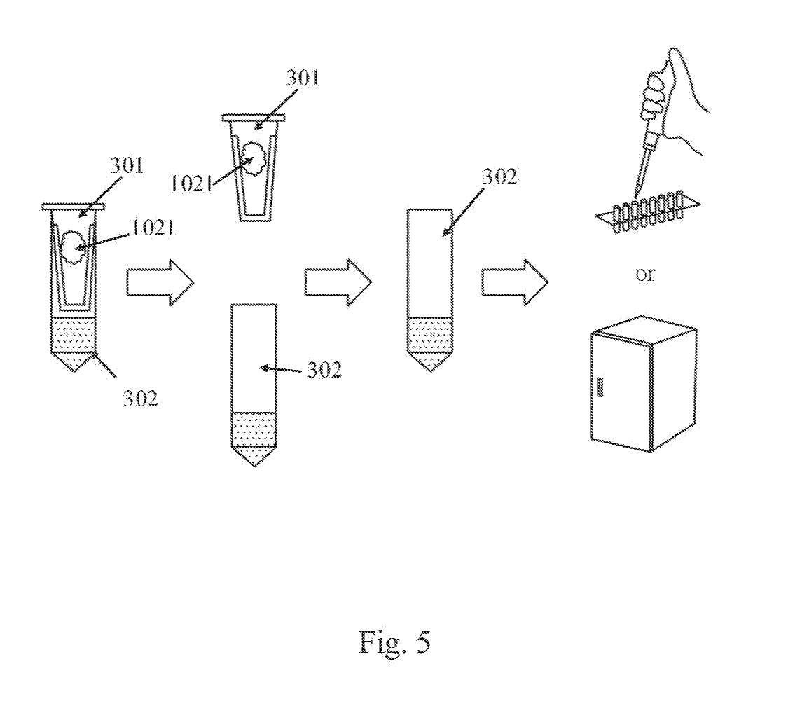

In another embodiment, the sampling adsorber 1021 of the sampling device 102 adsorbed with the body fluid may be processed with a centrifuge tube to obtain the body fluid adsorbed in the sampling adsorber 1021. Referring to FIG. 3, the sampling adsorber 1021 may be placed in an upper centrifuge tube 301 of the centrifuge tube first, and the sampling adsorber 1021 may be separated from the rod 1022, such as by breaking the rod 1022 or pulling the rod 1022 out of the sampling adsorber 1021. Then, as shown in FIG. 4, a buffer solution, for instance PBS (phosphate buffered saline), may be added into the upper centrifuge tube 301, and the upper centrifuge tube 301 is combined with a lower centrifuge tube 302. Subsequently, the combined centrifuge tube 30 is placed in a centrifuge 40 to perform centrifugation procedure. Next, with reference to FIG. 5, the body fluid mixed with the buffer solution will be obtained in the lower centrifuge tube 302 of the centrifuge tube 30 after the centrifugation procedure. Then, the upper centrifuge tube 301 is separated from the lower centrifuge tube 302, and the body fluid mixed with the buffer solution in the lower centrifuge tube 302 may be further utilized to test whether the test subject has a specific disease, for instance endometrial cancer, etc or to test the physiological condition, for example but be not limited to the ovulation cycle of woman, of the test subject by employing diagnostic devices, for example but be not limited to, paper-based diagnostic devices, lateral flow immunoassay devices, cotton-based diagnostic devices and so on, or may be refrigerated and reserved for other subsequent test operations.

The foregoing description is a preferred embodiment of the present invention. It should be appreciated that this embodiment is described for purposes of illustration only, not for limiting, and that numerous alterations and modifications may be practiced by those skilled in the art without departing from the spirit and scope of the present invention. It is intended that all such modifications and alterations are included insofar as they come within the scope of the present invention as claimed or the equivalents thereof.

What is claimed is:

1. A clinical specimen sampler, comprising:
a filtering device; and
a sampling device, said sampling device comprising a sampling adsorber and a rod, said sampling adsorber being connected with one end of said rod, said sampling adsorber being encompassed in said filtering device, wherein said sampling adsorber is utilized to absorb a body fluid secreted by an intima of an organ and said filtering device is employed to prevent a mucosal tissue of said intima of said organ from entering said sampling adsorber and enable said body fluid secreted by said intima of said organ to enter said sampling adsorber, so as to be absorbed by said sampling adsorber.

2. The device of claim 1, wherein said filtering device is bag-shaped.

3. The device of claim 2, wherein a binding wire or a quickly detachable fixing device is disposed around a bag opening of said filtering device to fix said filtering device onto said sampling adsorber of said sampling device.

4. The device of claim 1, wherein said filtering device comprises a filter screen.

5. The device of claim 1, wherein material of said filtering device comprises gauze, non-woven fabric or other material which can filter a mucosa and a secretion tissue.

6. The device of claim 1, wherein material of said sampling adsorber comprises sponge block, cotton ball or other material which can adsorb said body fluid.

7. The device of claim 1, wherein material of said rod comprises wooden rod, bamboo rod or plastic rod.

8. The device of claim 1, wherein said rod comprises a physically separable design to apply force to said physically separable design to separate said rod and said sampling adsorber.

9. The device of claim 8, wherein said physically separable design comprises a breaking notch.

10. The device of claim 1, further comprising a lubricant coated on an outer surface of said filtering device.

11. The device of claim 10, wherein said lubricant comprises vaseline.

12. The device of claim 1, wherein a size of a mesh of said filtering device is at least smaller than a size of said mucosal tissue.

13. A clinical specimen sampling method employing the clinical specimen sampler of claim 1, said method comprising:
rendering the sampling adsorber encompassed in the filtering device to contact a surface of the intima of the organ of a test subject to adsorb the body fluid secreted by the intima of the organ;
loosening a binding wire or a quickly detachable fixing device and separating the sampling device and the filtering device, such that the sampling device adsorbed with the body fluid is separated from the filtering device adhered with the mucosal tissue;
placing the sampling adsorber in an upper centrifuge tube of a centrifuge tube and separating the sampling adsorber from the rod;
adding a buffer solution into the upper centrifuge tube and combining the upper centrifuge tube with a lower centrifuge tube to form the centrifuge tube;
placing the combined centrifuge tube in a centrifuge to perform centrifugation procedure; and
separating the upper centrifuge tube from the lower centrifuge tube to obtain the body fluid mixed with the buffer solution in the lower centrifuge tube.

14. The method of claim 13, wherein said buffer solution comprises phosphate buffered saline.
